(19) United States
(12) Patent Application Publication
(10) Pub. No.: US 2026/0100280 A1
(43) Pub. Date: Apr. 9, 2026

(54) DIGITAL BIOMARKER OF DEMENTIA AND DEPRESSION

(71) Applicant: Pankaj Gupta, Edison, NJ (US)

(72) Inventor: Pankaj Gupta, Edison, NJ (US)

(21) Appl. No.: 18/908,349

(22) Filed: Oct. 7, 2024

(51) Int. Cl. G16H 50/20; G16H 10/60

(52) U.S. Cl. CPC G16H 50/20; G16H 10/60

(57) ABSTRACT

Embodiments directed to a computer-implemented diagnostic system and method to determine various diagnoses for different users, as well as symptom severity and onset of conditions associated with such diagnoses are described. In one example, a computer-implemented method of diagnosing health conditions for users includes identifying a defined linguistic pattern in digital biomarker data of a user. The method further includes prompting the user to perform a defined screening test based at least in part on identifying the defined linguistic pattern. The method further includes monitoring linguistic performance of the user while the user performs the defined screening test. The method further includes determining a diagnosis for the user based at least in part on the defined linguistic pattern, the linguistic performance, and a test result for the user from the defined screening test.

### BACKGROUND

The underlying pathology of certain medical and health conditions and diseases progresses silently in the brain for years before cognitive symptoms occur. For example, Alzheimer's disease (AD) is the most common cause of dementia and the underlying pathology progresses silently in the brain for 10-20 years before cognitive symptoms occur. This “pre-clinical” stage, and the subsequent mild cognitive impairment (MCI) stage of AD are critical to identify because pathology in the brain is minimal and therefore neuroprotective interventions, such as drug trials and risk reduction, have the greatest chance of success.

Dementia is typically diagnosed by a specialist doctor who performs a series of clinical assessments including obtaining a personal and informant history of the cognitive symptoms, a physical examination, pen and paper cognitive assessments, blood tests to rule out other mimics of dementia such as low vitamin B12 levels, and brain scans to assess for localized brain atrophy. This whole diagnostic process is time consuming, expensive, and somewhat subjective-relying heavily on the clinician's interpretation.

### SUMMARY

The present disclosure is directed to a computer-implemented diagnostic system and method to determine various diagnoses for different users, as well as symptom severity and onset of conditions associated with such diagnoses. The present disclosure describes a diagnostic framework that can be implemented by a client device or a combination of client devices such as a user's smartphone, wearable device, tablet, laptop, or another client device in some cases.

A client device can implement the diagnostic framework in many examples to determine a diagnosis for a user, severity of symptoms, and onset of conditions based in part on user data such as medical, health, healthcare, digital biomarker, and other data that have been collected by the device from the user, other devices, and digital data sources. The diagnostic framework can also be implemented by the device in many cases to make such determinations based in part on user speech and movement behaviors such as the user's linguistic patterns that have been learned by the device from monitoring the user over time. In many examples the device can further implement the diagnostic framework to make such determinations based in part on reference data collected by the device that are descriptive of various medical and health conditions, including data describing how to diagnose such conditions and determine their associated symptom severity and onset of conditions.

In some cases, the client device can further implement the diagnostic framework to perform one or more follow-up operations based at least in part on a diagnosis determined for a user. The diagnostic framework can be implemented by the device in one example to prompt the user to perform some follow-up action such as call or schedule an appointment with a medical provider, complete and submit benefits related documentation, or another action. The client device can implement the diagnostic framework in other cases to itself perform such a follow-up action on behalf of the user based at least in part on a diagnosis determined for the user.

Aspects and advantages of embodiments of the present disclosure will be set forth in part in the following description or can be learned from the description or through practice of the embodiments. Other aspects and advantages of embodiments of the present disclosure will become better understood with reference to the appended claims and the accompanying drawings, all of which are incorporated in and constitute a part of this specification. The drawings illustrate example embodiments of the present disclosure and, together with the description, serve to explain the related concepts of the present disclosure.

In one example embodiment, a computer-implemented method of diagnosing health conditions for users includes identifying a defined linguistic pattern in digital biomarker data of a user. The method further includes prompting the user to perform a defined screening test based at least in part on identifying the defined linguistic pattern. The method further includes monitoring linguistic performance of the user while the user performs the defined screening test. The method further includes determining a diagnosis for the user based at least in part on the defined linguistic pattern, the linguistic performance, and a test result for the user from the defined screening test.

In another example embodiment, a computing device includes a memory device to store computer-readable instructions thereon and at least one processing device configured through execution of the computer-readable instructions to identify a defined linguistic pattern in digital biomarker data of a user. The at least one processing device is further configured to prompt the user to perform a defined screening test based at least in part on identifying the defined linguistic pattern. The at least one processing device is further configured to monitor linguistic performance of the user while the user performs the defined screening test. The at least one processing device is further configured to determine a diagnosis for the user based at least in part on the defined linguistic pattern, the linguistic performance, and a test result for the user from the defined screening test.

In yet another example embodiment, a computer-implemented method of diagnosing health conditions for users includes identifying a defined linguistic pattern in digital biomarker data of a user. The method further includes implementing a defined screening test to be performed by the user based at least in part on identifying the defined linguistic pattern. The method further includes monitoring linguistic performance of the user during the defined screening test. The method further includes determining a diagnosis for the user based at least in part on the defined linguistic pattern, the linguistic performance, and a test result for the user from the defined screening test.

### BRIEF DESCRIPTION OF THE DRAWINGS

Many aspects of the present disclosure can be better understood with reference to the following figures. The components in the figures are not necessarily to scale, with emphasis instead being placed upon clearly illustrating the concepts of the disclosure. Moreover, repeated use of reference characters or numerals in the figures is intended to represent the same or analogous features, elements, or operations across different figures. Repeated description of such repeated reference characters or numerals is omitted for brevity.

FIG. 1 illustrates a block diagram of an example environment according to various aspects and embodiments of the present disclosure.

FIG. 2 illustrates a block diagram of an example computing device of the environment shown in FIG. 1 according to various aspects and embodiments of the present disclosure.

FIG. 3 illustrates a flow diagram of an example computer-implemented method according to various aspects and embodiments of the present disclosure.

FIG. 4 illustrates a flow diagram of another example computer-implemented method according to various aspects and embodiments of the present disclosure.

### DETAILED DESCRIPTION

With populations aging, the number of people with dementia worldwide is expected to triple to 152 million by 2050. Alzheimer's disease (AD) is the most common cause of dementia. Seventy percent of dementia cases are due to AD pathology and there is a 10-20 year “pre-clinical” period before significant cognitive decline occurs. This pre-clinical stage, and the subsequent mild cognitive impairment (MCI) stage of AD are critical to identify because pathology in the brain is minimal and therefore neuroprotective interventions, such as drug trials and risk reduction, have the greatest chance of success. Early modification of lifestyle (e.g., healthcare to prevent obesity) and medical risk factors (e.g., hypertension) could prevent 40% of dementia cases.

Dementia is associated with a significant personal cost to individuals and their families, a substantial healthcare burden, and costs of more than US $1 trillion annually. Using objective biomarkers to detect AD and other dementias at an early stage in conjunction with risk factor modification could prevent many dementia cases, and drug trials could have greater chances of success if participants were to be recruited at an earlier stage.

Currently, detection of dementia is largely by pen and paper cognitive tests but these are time consuming and insensitive to the pre-clinical phase. Specialist brain scans and body fluid biomarkers can detect the earliest stages of dementia but are too invasive or expensive for widespread use. With the advancement of technology, Artificial Intelligence (AI) shows promising results in assisting with detection of early-stage dementia.

The medical and health professional community lacks accessible population-level tests to detect pre-clinical AD, MCI, or the earliest stages of AD-before significant cognitive and functional decline occur. Currently, AD is usually diagnosed when cognitive symptoms such as memory impairment appear, after more than 20 years of progressive brain pathology. Symptoms of AD gradually progress to language, reasoning, and planning impairments, and there may also be psychiatric symptoms such as hallucinations, behavior changes such as apathy or agitation, and physical changes such as falls. Other common causes of dementia include frontotemporal dementia (FTD), Lewy body dementia (LBD), and vascular dementia.

Dementia is typically diagnosed by a specialist doctor who performs a series of clinical assessments including obtaining a personal and informant history of the cognitive symptoms, a physical examination, pen and paper cognitive assessments, blood tests to rule out other mimics of dementia (e.g., low vitamin B12 levels), and brain scans to assess for localized brain atrophy. This whole diagnostic process is time consuming, expensive and somewhat subjective—relying heavily on the clinician's interpretation.

In the last decade, there have been advances in developing new specialist tests to directly detect the pathological proteins of dementia through brain scans and spinal fluid and blood tests; however, these biomarkers remain too invasive, costly or specialist to be widely accessible in clinical practice. Recent developments in computer science, especially Artificial Intelligence (AI), also offer a potential solution to this global problem. They provide the technologies that could aid development of new efficient and accessible methods to assist in detecting the earliest stages of AD and other dementias. A number of reviews have looked at how AI may assist with certain tests and investigations used in the diagnostic work-up for dementia (e.g., MRI scans, cognitive tests). However, there has not been a recent review of how AI assists dementia screening across a range of tests.

Recently, various AI-based digital biomarkers have been introduced to assist with the detection of dementia—either through detecting functional changes (e.g., cognitive, movement or speech impairments) or through detecting pathological abnormalities on brain scans. AI-based digital biomarkers can provide improved accuracy through their capability of capturing additional features from a large amount of data. This brings out more objective inference compared with clinicians'manually analyzed results. Furthermore, AI provides an automated analysis process—both in terms of time and cost efficiencies. Recently introduced AI-based tests include computerized cognitive tests, computer-assisted interpretation of brain scans, and movement and speech-analysis tests.

The neuropsychological profile of people with Mild Cognitive Impairment (MCI) and Alzheimer's Disease (AD) dementia includes a history of decline in memory and other cognitive domains, including language. While language impairments have been well described in AD, language features of MCI are less understood. A potentially sensitive measure of language in MCI is analysis of connected speech.

Connected speech analysis is the study of an individual's spoken discourse, usually elicited by a target stimulus, the results of which can facilitate understanding of how language deficits typical of MCI and AD manifest in everyday communication. Among discourse genres, picture description is a constrained task that relies less on episodic memory and more on semantic knowledge and retrieval, within the cognitive demands of a communication context.

Language deficits in AD dementia have been well documented and the language profile of adults with AD typically is characterized by “empty speech,” referring to word retrieval deficits that result in the use of circumlocutions, nonspecific language, and an overabundance of words conveying limited ideas. In the moderate to severe stages of disease, communication skills degrade further with deficits in both production and comprehension of language, reflected in communication breakdowns in everyday interactions and increased frustration that may result in challenging behaviors. Often the end stage of AD is characterized by a complete lack of verbal communication, and the person with AD becomes socially disengaged.

In view of the importance of early detection of various medical and health conditions and diseases, and to address the aforementioned problems with diagnostic processes in general and specific to dementia, embodiments herein include a computer-implemented diagnostic system and method to determine various diagnoses for different users, as well as symptom severity and onset of conditions associated with such diagnoses. The embodiments can implement the diagnostic framework of the present disclosure in many cases using a client device or a combination of client devices such as a user's smartphone, wearable device, tablet, laptop, or another client device.

A client device in one embodiment can implement the diagnostic framework to determine a diagnosis for a user, severity of symptoms, and onset of conditions based in part on user data collected by the device, user speech and movement behaviors learned by the device, and reference medical and health data collected by the device. The reference medical and health data being descriptive of various medical and health conditions, as well as how to diagnose such conditions and determine their associated symptom severity and onset of conditions.

The embodiments implement automated, objective, accurate, and cost-effective methods to not only analyze the outcome at the end of a screening test but also the cognitive performance of the individual completing the test while the individual is in the process of completing the test. The embodiments utilize cost-effective objective and accessible digital biomarkers to detect various medical and health conditions and diseases such as AD, and other types of dementia, at a population level. By incorporating machine learning technologies such as speech pattern analysis, the embodiments can extract additional features, which improves the speed and accuracy of screening processes for various conditions or diseases such as dementia. The embodiments can also accurately diagnose depression and psychosis in some cases, as well as measure severity of symptoms and predict onset of mental health conditions associated with such diagnoses.

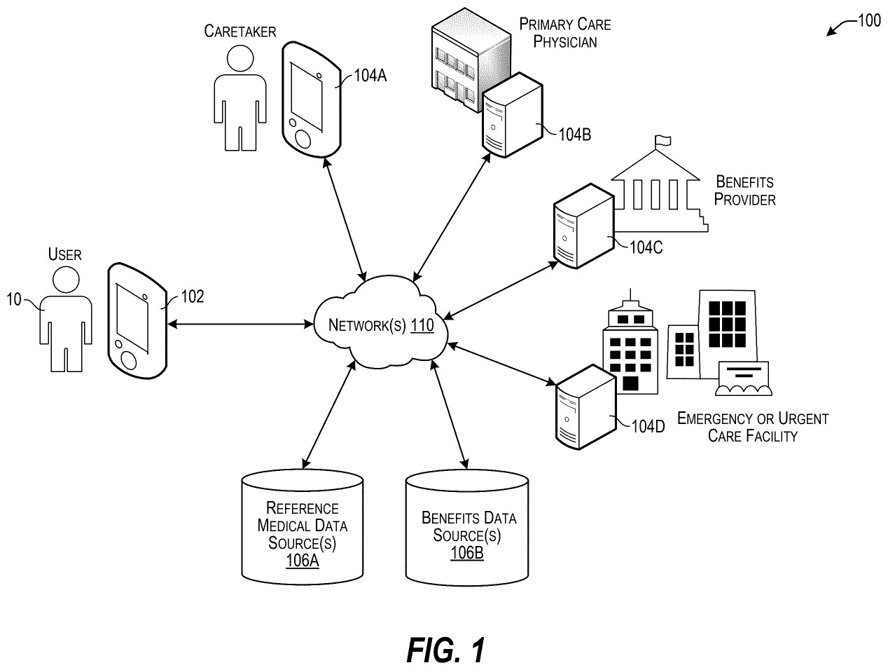

For context, FIG. 1 illustrates a block diagram of an example environment 100 according to various aspects and embodiments of the present disclosure. The environment 100 can be a computing environment in which various types of computing operations can be performed, among other operations. The environment 100 is illustrated as a representative example, and the diagnostic framework concepts described herein are not limited to use with any particular type of computing environment.

The environment 100 includes a computing device 102, one or more remote computing devices 104, and one or more data sources 106, among other components. The computing device 102, the remote computing devices 104, and the data sources 106 are coupled to one another in this example by way of one or more networks 110. The remote computing devices 104 include remote computing devices 104A, 104B, 104C, 104D in the example shown, although the environment 100 may include a different number or type of remote computing devices in other examples. The data sources 106 include a reference medical data source 106A and a benefits data source 106B in this example, although the environment 100 may include a different number or type of data sources in other examples.

The computing device 102 is associated with (e.g., owned by, operated by) a user 10 such as a human, and each of the remote computing devices 104 is associated with (e.g., owned by, operated by) a third-party entity in this example. The remote computing device 104A is associated with a caretaker of the user 10 and the remote computing device 104B is associated with a primary care physician of the user 10. The remote computing device 104C is associated with a benefits provider such as a health benefits provider, an insurance benefits provider, a retirement benefits provider, a social security benefits provider (e.g., United States Social Security Administration), another benefits provider, or any combination thereof. The remote computing device 104D is associated with an emergency or urgent care facility such as a hospital, an emergency room or department, an urgent care center, another facility, or any combination thereof.

The computing device 102 and any or all of the remote computing devices 104 can each be embodied or implemented as one or more of a server computing device, a client computing device, a general-purpose computer, a special-purpose computer, a virtual machine, a supercomputer, a laptop, a tablet, a smartphone, a wearable device, or another type of computing device that can be configured and operable to perform various operations described herein. A detailed description of the computing device 102 and the operations it can perform is provided below.

The reference medical data source 106A can be embodied as a digital data source that can be accessed by the computing device 102 such as an online website, repository, database, another type of digital data source, or any combination thereof. The reference medical data source 106A can include a plurality of digital resources having data and information that are indicative of or describe various medical and health related topics, subjects, studies, experimentations, innovations, treatments, case studies, other such data or information, or any combination thereof. The reference medical data source 106A can be embodied in many examples as a collection of digital publications, articles, books, journals, illustrations, videos, other digital forms of such data and information, or any combination thereof.

As described in examples herein, the reference medical data source 106A can include various medical and health data and information that can be used by the computing device 102 as training data to lean various indicators and precursors associated with and at least partly indicative of different medical conditions and diagnoses. For instance, the reference medical data source 106A can include a plurality of different reference linguistic patterns, digital biomarker data, linguistic performance data, screening test result data, and other reference data that are at least partly indicative of different medical or health conditions and diagnoses. The computing device 102 can be configured to use such data to learn to identify certain indicators and precursors associated with a multitude of different medical or health conditions and diagnoses. The medical and health data and information in the reference medical data source 106A can also be used by the computing device 102 as reference material. For instance, when predicting diagnoses for the user 10 as described further herein the computing device 102 can be configured to compare corresponding data obtained from the user 10 to reference linguistic patterns, reference digital biomarker data, reference linguistic performance data, and reference screening test result data among other data in the reference medical data source 106A that are at least partly indicative of different medical or health diagnoses.

The benefits data source 106B can also be embodied as a digital data source that can be accessed by the computing device 102 such as an online website, repository, database, another type of digital data source, or any combination thereof. The benefits data source 106B can include a plurality of digital resources having data and information that are indicative of or describe various medical and health benefits plans and corresponding benefits provided by different benefits providers and agencies. For instance, such data and information can be indicative of or describe plan qualification requirements, plan application completion and submittal processes, benefits claims submittal processes, benefits distribution procedures, and other content associated with different benefits plans and corresponding benefits provided by different benefits providers and agencies. The benefits data source 106B can be embodied in many examples as a collection of digital publications, articles, books, journals, illustrations, videos, other digital forms of such data and information, or any combination thereof. The data and information included in the benefits data source 106B in some cases can include digital communication links (e.g., hyperlinks) that allow the computing device 102 to communicate by way of the networks 110 with a computing device associated with a benefits provider or agency. In one example, the benefits data source 106B can include data and information that are indicative of or describe various medical and health benefits plans and corresponding benefits provided by the benefits provider associated with the remote computing device 104C.

As described in examples herein, the benefits data source 106B can include various medical and health benefits data and information that can be used by the computing device 102 as training data to learn about various benefits available to individuals across different medical diagnoses, as well as the processes for acquiring or implementing the distribution of such benefits. In some examples, the computing device 102 can use such medical and health benefits data and information to learn how the user 10 can acquire or implement the distribution of various benefits using the computing device 102. In other examples, the computing device 102 can use such benefits data and information to learn how the computing device 102 can acquire or implement the distribution of various benefits on behalf of the user 10.

The networks 110 can include, for instance, the Internet, intranets, extranets, wide area networks (WANs), local area networks (LANs), wired networks, wireless networks (e.g., cellular, WiFi®), cable networks, satellite networks, other suitable networks, or any combinations thereof. The computing device 102, the remote computing devices 104, and the data resources 106 can communicate data with one another over the networks 110 using any suitable systems interconnect models and/or protocols. Example interconnect models and protocols include hypertext transfer protocol (HTTP), simple object access protocol (SOAP), representational state transfer (REST), real-time transport protocol (RTP), real-time streaming protocol (RTSP), real-time messaging protocol (RTMP), user datagram protocol (UDP), internet protocol (IP), transmission control protocol (TCP), and/or other protocols for communicating data over the networks 110, without limitation. Although not illustrated, the networks 110 can also include connections to any number of other network hosts, such as website servers, file servers, networked computing resources, databases, data stores, or other network or computing architectures in some cases.

Among other operations, the computing device 102 can be configured to monitor the user 10 over time to determine a medical or health diagnosis for the user 10, determine severity of one or more symptoms associated with such a diagnosis, and predict an onset of at least one medical or health condition associated with the diagnosis in some cases. As described further herein, the computing device 102 can determine a diagnosis for the user 10, the severity of symptoms, and onset of conditions based in part on information the computing device 102 learns from monitoring the user 10 over time and in part on information the computing device 102 learns about various medical and health conditions, including how to diagnose such conditions and determine their associated symptom severity and onset of conditions.

The computing device 102 can be configured to monitor the user 10 over time by periodically or continuously using one or more input/output devices (e.g., camera, microphone, keyboard, lights, speaker) and one or more physiological sensors (e.g., electrocardiogram (ECG) sensor, electroencephalogram (EEG) sensor, respiration sensor) in some cases to capture different types of digital biomarker data of the user 10 during different activities. The digital biomarker data can include, but is not limited to, at least one of audio data of the user 10 speaking or moving, video data of the user 10 speaking or moving, image data of the user 10 speaking or moving, textual data typed by the user 10, and physiological data of the user 10 captured while the user speaks or moves.

The computing device 102 can be configured to learn linguistic or other behavior patterns of the user 10 over time using the digital biomarker data captured from the user 10 in conjunction with various medical and health data and information available in the reference medical data source 106A. The computing device 102 can also be configured to use the medical and health data and information in the reference medical data source 106A to learn to identify certain indicators and precursors associated with different medical and health conditions and diagnoses. For instance, the computing device 102 can use such data and information to learn to identify particular linguistic patterns, digital biomarker data, linguistic performance data, and screening test result data that are at least partly indicative of different medical and health conditions and diagnoses.

To determine a diagnosis for the user 10, as well as the severity of symptoms and onset of conditions associated with the diagnosis in one example, the computing device 102 can be configured to identify a defined linguistic pattern in digital biomarker data of the user 10. For instance, the computing device 102 can identify a certain linguistic pattern that it has learned to be at least partly indicative of a particular medical or health condition or diagnosis. The computing device 102 can identify the defined linguistic pattern in static or historical digital biomarker data of the user 10. For example, the computing device 102 can identify the defined linguistic pattern in digital biomarker data that has been captured from the user 10 at a particular moment in time while the user 10 was performing some activity, or in historical digital biomarker data captured from the user 10 over some period of time while the user 10 performed various activities.

The computing device 102 can be configured to then prompt the user 10 to perform a defined screening test based at least in part on identifying the defined linguistic pattern as described. For instance, the computing device 102 can generate and provide at least one notification to the user 10 that includes a recommendation for the user 10 to complete one or more specific screening tests. For example, the computing device 102 can generate and provide at least one of a push notification, a text message, an electronic-mail (e-mail) message, an audio message or alert, a video message or alert, or another type of notification that includes a recommendation for the user 10 to complete one or more specific screening tests. Examples of the defined screening test include, but are not limited to, a medical or health screening task or process, a cognitive screening task or process, a verbal fluency screening task or process, a semantic fluency screening task or process, a phonemic fluency screening task or process, a computer-implemented cognitive test, a movement and speech analysis test, another screening test, or any combination thereof.

The computing device 102 can also be configured to monitor linguistic performance of the user 10 while the user 10 performs the defined screening test. For instance, the computing device 102 can use input/output devices (also “I/O devices”) and physiological sensors to capture various data from the user 10 while the user 10 performs the defined screening test. The computing device 102 can use such devices and sensors to capture at least one of video data, image data, audio data, text data, physiological data, or other data corresponding to the user 10 while the user 10 performs the defined screening test in many examples. The computing device 102 can analyze the captured data to observe the linguistic behavior of the user 10 during the defined screening test. For instance, the computing device 102 can analyze the captured data to observe how the user 10 speaks and moves while actively completing the defined screening test.

The computing device 102 can further be configured to determine a diagnosis for the user 10 based at least in part on the defined linguistic pattern, the linguistic performance of the user 10 while performing the defined screening test, and a test result for the user 10 obtained from completing the defined screening test. For instance, the computing device 102 can determine that at least one of the defined linguistic pattern, the linguistic performance, or the test result includes or is indicative of one or more language features or language deficits that are at least partly indicative of the diagnosis. Examples of the diagnosis include, but are not limited to, a medical or health condition, a neurodegenerative condition, a neurologic condition, a psychiatric condition, a geriatric condition, a prodromal dementia condition, a prodromal depression condition, a psychosis condition, an anxiety condition, an attention-deficit/hyperactivity disorder (ADHD) condition, a mild cognitive impairment condition, an Alzheimer's condition, a prodromal dementia condition, a dementia condition, a prodromal depression condition, a depression condition, another condition, or any combination thereof.

The computing device 102 can also be configured to determine severity of one or more symptoms associated with the diagnosis in some cases based at least in part on one or more of the defined linguistic pattern, the digital biomarker data, the linguistic performance, or the test result. Additionally, the computing device 102 can be configured to predict an onset of one or more conditions associated with the diagnosis in some examples based at least in part on one or more of the defined linguistic pattern, the digital biomarker data, the linguistic performance, or the test result. To determine a diagnosis for the user 10, as well as the severity of symptoms and onset of conditions associated with the diagnosis in some examples, the computing device 102 can compare data collected or learned from the user 10 to reference data that are at least partly indicative of the diagnosis, the severity of symptoms, or onset of conditions. For instance, the computing device 102 can compare learned linguistic patterns, digital biomarker data, linguistic performances, and screening test results of the user 10 to reference linguistic patterns, reference digital biomarker data, reference linguistic performance data, and reference screening test result data, respectively, that are located in the reference medical data source 106A and are at least partly indicative of the diagnosis, the severity of symptoms, or onset of conditions. The computing device 102 can also compare recently captured digital biomarker data of the user 10 in some cases to historical digital biomarker data of the user 10 to identify any change in the data overtime which may be at least partly indicative of the diagnosis, the severity of symptoms, or onset of conditions.

Based on determining a diagnosis, the severity of symptoms, or onset of conditions in some examples, the computing device 102 can further be configured to prompt the user 10 to perform one or more follow-up actions described herein such as contacting or scheduling an appointment with the user's PCP. In other examples, the computing device 102 can be configured to itself perform one or more follow-up operations on behalf of the user 10 based at least in part on determining a diagnosis, the severity of symptoms, or onset of conditions. For instance, the computing device 102 can be configured to communicate on behalf of the user 10 with any of the third-party entities associated with any of the remote computing devices 104. The computing device 102 can be configured in one example to communicate with the remote computing device 104C on behalf of the user 10 to complete and submit various documentation for the user 10 in connection with applying for benefits coverage or submitting a claim for benefits distribution from the benefits provided associated with the remote computing device 104C.

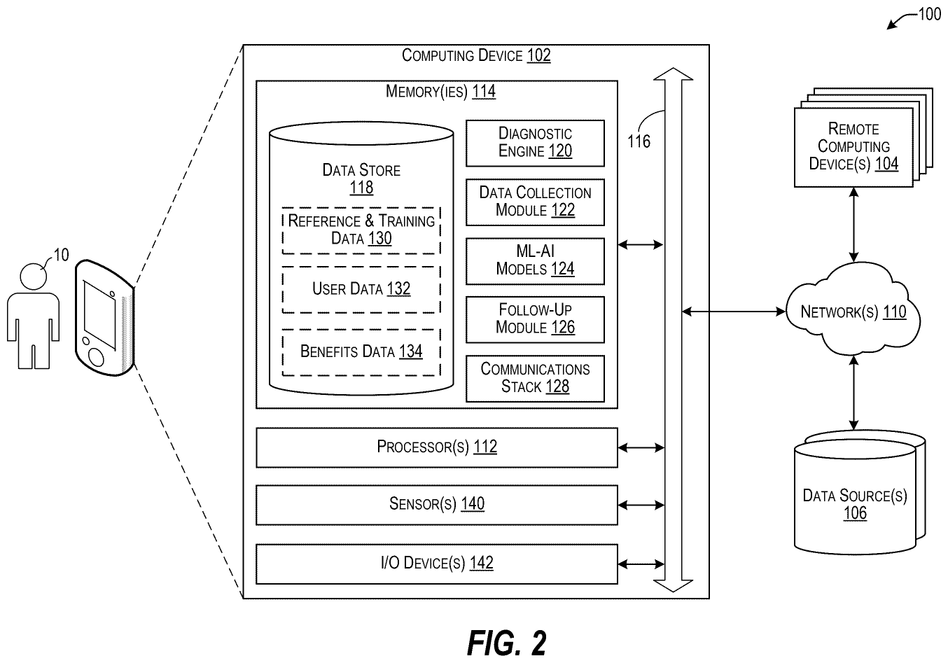

FIG. 2 illustrates a block diagram of the computing device 102 shown in FIG. 1 according to various aspects and embodiments of the present disclosure. To determine a diagnosis for the user 10, as well as the severity of symptoms and onset of conditions associated with the diagnosis in various examples, the computing device 102 can include at least one processing and memory system. In the example depicted in FIGS. 1 and 2, the computing device 102 includes at least one processor 112 and at least one memory 114, both of which are communicatively coupled, operatively coupled, or both, to a local interface 116. The memory 114 includes a data store 118, a diagnostic engine 120, a data collection module 122, machine learning and artificial intelligence (ML-AI) models 124 (also “ML-AI models 124”), a follow-up module 126, and a communications stack 128 in the example shown. The computing device 102 is coupled to the networks 110 by way of the local interface 116 in this example. The computing device 102 further includes one or more sensors 140 and one or more input/output (I/O) devices 142 (also “I/O devices 142”) coupled to the local interface 116 in the example shown. The computing device 102 can also include other components that are not illustrated in FIG. 1. The data store 118 in this example includes reference and training data 130, user data 132, and benefits data 134.

The processor 112 can be embodied as or include any processing device (e.g., a processor core, a microprocessor, an application specific integrated circuit (ASIC), a field-programmable gate array (FPGA), a controller, a microcontroller, or a quantum processor) and can include one or multiple processors that can be operatively connected. In some examples, the processor 112 can include one or more complex instruction set computing (CISC) microprocessors, one or more reduced instruction set computing (RISC) microprocessors, one or more very long instruction word (VLIW) microprocessors, or one or more processors that are configured to implement other instruction sets.

The memory 114 can be embodied as one or more memory devices and can store data and software or executable-code components executable by the processor 112. For example, the memory 114 can store executable-code components associated with the diagnostic engine 120, the data collection module 122, the ML-AI models 124, the follow-up module 126, and the communications stack 128 for execution by the processor 112. The memory 114 can also store data such as the data described below that can be stored in the data store 118, among other data. For instance, the memory 114 can also store data indicative of at least one of the reference and training data 130, the user data 132, or the benefits data 134.

The memory 114 can store other executable-code components for execution by the processor 112. For example, an operating system can be stored in the memory 114 for execution by the processor 112. Where any component discussed herein is implemented in the form of software, any one of a number of programming languages can be employed such as, for example, C, C++, C #, Objective C, JAVA®, JAVASCRIPT®, Perl, PHP, VISUAL BASIC®, PYTHON®, RUBY, FLASH®, or other programming languages.

As discussed above, the memory 114 can store software for execution by the processor 112. In this respect, the terms “executable” or “for execution” refer to software forms that can ultimately be run or executed by the processor 112, whether in source, object, machine, or other form. Examples of executable programs include, for instance, a compiled program that can be translated into a machine code format and loaded into a random access portion of the memory 114 and executed by the processor 112, source code that can be expressed in an object code format and loaded into a random access portion of the memory 114 and executed by the processor 112, source code that can be interpreted by another executable program to generate instructions in a random access portion of the memory 114 and executed by the processor 112, or other executable programs or code.

The local interface 116 can be embodied as a data bus with an accompanying address/control bus or other addressing, control, and/or command lines. In part, the local interface 116 can be embodied as, for instance, an on-board diagnostics (OBD) bus, a controller area network (CAN) bus, a local interconnect network (LIN) bus, a media oriented systems transport (MOST) bus, ethernet, or another network interface.

The data store 118 can include data for the computing device 102 such as, for instance, one or more unique identifiers for the computing device 102, digital certificates, encryption keys, session keys and session parameters for communications, and other data for reference and processing. The data store 118 can also store computer-readable instructions for execution by the computing device 102 via the processor 112, including instructions for the diagnostic engine 120, the data collection module 122, the ML-AI models 124, the follow-up module 126, and the communications stack 128.

The reference and training data 130 can include and be indicative of various medical and health data and information that can be obtained by the computing device 102 from the reference medical data source 106A. The reference and training data 130 can be used by the computing device 102 as training data in some examples such as when learning to identify linguistic patterns that are at least partly indicative of different diagnoses. In other examples, the reference and training data 130 can be used as reference material such as when the computing device 102 compares the user data 132 to reference or baseline medical and health data and information that is at least partly indicative of one or more diagnoses. The reference and training data 130 can also include and be indicative of various benefits data and information in some cases such as medical and health benefits data and information that can be obtained by the computing device 102 from the benefits data source 106B and used as reference or training data.

The user data 132 can include and be indicative of various data corresponding to and indicative of the user 10. The computing device 102 can be configured to obtain the user data 132 from at least one of the remote computing devices 104, the data sources 106, or another device or data source in some examples, capture it from the user 10 in others, or learn it over time in some cases. Example user data 132 can include, but are not limited to, identification data or information (e.g., name, driver's license number, social security number), contact data or information (e.g., address, phone number, e-mail address), digital biomarker data or information (e.g., video data, image data, audio data, physiological data), linguistic performance data or information (e.g., video data, image data, audio data, physiological data, and other data of the user 10 captured while the user 10 performs a defined screening test), screening test results data or information (e.g., results data or information for the user 10 obtained upon completion of screening tests), medical or health data or information (e.g., age, weight, height, medical history, existing diagnoses, medication history, existing medications and corresponding dosages), learned behavior patterns data or information (e.g., learned linguistic patterns, linguistic performances, and screening test results of the user 10), other data or information corresponding to and indicative of the user 10, or any combination thereof.

The benefits data 134 can include and be indicative of one or more benefits plans associated with and corresponding to the user 10. The computing device 102 can be configured to obtain the benefits data 134 from at least one of the remote computing devices 104, the data sources 106, or another device or data source in some examples, capture it from the user 10 in others, or learn it over time in some cases. Example benefits data 134 can include, but are not limited to, benefits plan identification, corresponding benefits, and provider data or information for any historical, existing, or pending benefits plans associated with the user 10 (e.g., applied for or enrolled in by the user 10 or on behalf of the user 10).

Each of the diagnostic engine 120, the data collection module 122, the ML-AI models 124, and the follow-up module 126 can be embodied as one or more software applications or services executing on the computing device 102. The diagnostic engine 120 can be executed by the processor 112 as described in examples herein to determine a diagnosis, symptom severity, and onset of conditions for the user 10 using the data collection module 122, the ML-AI models 124, the reference and training data 130, the user data 132, the sensors 140, and the I/O devices 142, among other components.

The data collection module 122 can be executed by the processor 112 to collect or capture various types of data from at least one of the user 10, the remote computing devices 104, or the data sources 106. For instance, the data collection module 122 can be configured to collect the reference and training data 130 from the reference medical data source 106A and the benefits data source 106B, the benefits data 134 from the remote computing device 104C, and the user data 132 from the user 10. For example, the data collection module 122 can capture the user data 132 using at least one of the sensors 140 of the I/O devices 142. The data collection module 122 can be configured to collect and store any or all of the reference and training data 130, the user data 132, or the benefits data 134 in the data store 118 on a periodic or continuous basis.

The ML-AI models 124 can be executed by the processor 112 to learn one or more linguistic patterns of the user 10 over time based at least in part on the user data 132 such as historical digital biomarker data collected by the data collection module 122 from the user 10 at different times and during different activities. The ML-AI models 124 can also be executed by the processor 112 to learn to identify various linguistic patterns that are at least partly indicative of different diagnoses. For example, the ML-AI models 124 can use the reference and training data 130 to learn a multitude of reference medical and health data that are at least partly indicative of different diagnoses, as well as severity of symptoms and onset of conditions associated with such diagnoses. For instance, the ML-AI models 124 can use the reference and training data 130 to learn to identify certain linguistic patterns, digital biomarker data, linguistic performance data, and screening test result data that are at least partly indicative of different diagnoses and to further determine the severity of symptoms and predict onset of conditions associated with such diagnoses. The ML-AI models 124 can include various machine learning and artificial intelligence models such as one or more neural networks, deep neural networks, convolutional neural networks (CNN), large language models, pattern and object recognition models, speech or linguistic recognition models, speech or linguist pattern recognition models, speech-to-text models, other models, or any combination thereof.

The communications stack 128 can include software and hardware layers to implement data communications such as, for instance, Bluetooth®, Bluetooth® Low Energy (BLE), WiFi®, cellular data communications interfaces, or a combination thereof. Thus, the communications stack 128 can be relied upon by the computing device 102 to establish cellular, Bluetooth®, WiFi®, and other communications channels with the networks 110 and with at least one of the remote computing devices 104 or the data resources 106.

The communications stack 128 can include the software and hardware to implement Bluetooth®, BLE, and related networking interfaces, which provide for a variety of different network configurations and flexible networking protocols for short-range, low-power wireless communications. The communications stack 128 can also include the software and hardware to implement WiFi® communication, and cellular communication, which also offers a variety of different network configurations and flexible networking protocols for mid-range, long-range, wireless, and cellular communications. The communications stack 128 can also incorporate the software and hardware to implement other communications interfaces, such as X10®, ZigBee®, Z-Wave®, and others.

The communications stack 128 can be configured to communicate various data or information amongst the computing device 102, the remote computing devices 104, and the data resources 106. Examples of such data or information can include, but is not limited to, at least one of data indicative of the reference and training data 130, the user data 132, the benefits data 134, other data, or any combination thereof.

The sensors 140 can be embodied and configured to capture various types of physiological data from the user 10. For instance, the sensors 140 can include, but are not limited to, an electrocardiogram (ECG) sensor to capture heart rate data, an electroencephalogram (EEG) sensor to capture brain activity data, an electromyogram (EMG) sensor to capture muscle activity data, a galvanic skin response (GSR) sensor to capture skin conductivity data, a respiration sensor to capture breathing rate data, a photoplethysmography (PPG) to capture blood volume change data, a skin temperature sensor to capture skin temperature data, and one or more eye tracking sensors to capture pupil dilation data.

The I/O devices 142 can be embodied and configured to capture and communicate various types of data from and with the user 10. For instance, the I/O devices 142 can include, but are not limited to, a camera, a display, a monitor, a screen, a microphone, a speaker, a keyboard, a mouse, a mouse pad, a haptic device, another I/O device, or any combination thereof. The I/O devices 142 can capture at least one of video data, image data, audio data, vibration data, text data, another type of user data, or any combination thereof from the user 10. The I/O devices 142 can be used to capture such data on a periodic or continuous basis.

Referring to FIGS. 1 and 2, the computing device 102 can implement the diagnostic engine 120 to perform various operations described in examples herein. For instance, the computing device 102 can implement the diagnostic engine 120 to determine a medical or health diagnosis for the user 10, determine severity of one or more symptoms associated with such a diagnosis, and predict an onset of at least one medical or health condition associated with the diagnosis. The diagnostic engine 120 can determine such a diagnosis, symptom severity, and onset of conditions for the user 10 by implementing the data collection module 122 and the ML-AI models 124 to perform their respective operations using the reference and training data 130, the user data 132, the sensors 140, and the I/O devices 142, among other components.

The diagnostic engine 120 can be configured in one example to determine a diagnosis at least in part for the user 10 using the ML-AI models 124 to identify a defined linguistic pattern in digital biomarker data of the user 10 that has been captured using the data collection module 122. The diagnostic engine 120 can be further configured in another example to determine at least in part a diagnosis for the user 10 using the ML-AI models 124 to identify a defined linguistic pattern in linguistic performance data of the user 10 that has been captured using the data collection module 122 while the user 10 performs a defined screening test. The diagnostic engine 120 can also be configured in yet another example to determine at least in part a diagnosis for the user 10 using the ML-AI models 124 to identify a defined linguistic pattern in a test result for the user 10 obtained using the data collection module 122 upon completion by the user 10 of a defined screening test. For instance, the test result can include data or information that are at least partly indicative of the defined linguistic pattern or the diagnosis.

The diagnostic engine 120 can be configured in many cases to prompt the user 10 to perform one or more follow-up actions based at least in part on identifying a defined linguistic pattern in any of the user data 132. For instance, the diagnostic engine 120 can use the follow-up module 126 to prompt (e.g., by way of the I/O devices 142, text messaging, push notification) the user 10 to perform a defined screening test based at least in part on identifying a defined linguistic pattern in any of the user data 132. Additionally, the diagnostic engine 120 can be configured in many cases to itself implement a defined screening test for the user 10 to complete based at least in part on identifying a defined linguistic pattern in any of the user data 132. For instance, the diagnostic engine 120 can use the follow-up module 126 to implement a defined screening test for the user 10 to complete using the computing device 102. The diagnostic engine 120 can also use the data collection module 122, the sensors 140, and the I/O devices 142 to monitor the linguistic patterns or other behavior or performance of the user 10 while the user 10 is performing the defined screening test.

The diagnostic engine 120 can be configured in many cases to then use the ML-AI models 124 to analyze at least one of audio data, video data, or physiological data of the user 10 that are captured while the user 10 performs the defined screening test. In some cases, the diagnostic engine 120 can use the ML-AI models 124 to compare linguistic patterns of the user 10 observed during the screening test and over time to reference linguistic patterns that are at least partly indicative of different diagnoses. The diagnostic engine 120 can also use the ML-AI models 124 to compare digital biomarker data of the user 10 captured during the screening test or over time to reference digital biomarker data that are at least partly indicative of different diagnoses. The diagnostic engine 120 can also use the ML-AI models 124 to compare linguistic performance data of the user 10 observed during the screening test or over time to reference linguistic performance data that are at least partly indicative of different diagnoses. The diagnostic engine 120 can also compare screening test result data for the user 10 obtained from the screening test or other tests to reference screening test result data that are at least partly indicative of different diagnoses. Based at least in part on the aforementioned analysis and comparisons performed by the diagnostic engine 120 using the ML-AI models 124, the diagnostic engine 120 can ultimately determine a diagnosis for the user 10, as well as severity of symptoms and onset of conditions associated with the diagnosis.

The diagnostic engine 120 can further be configured in many cases to prompt the user 10 to perform one or more follow-up actions based at least in part on determining a diagnosis for the user 10. For instance, the diagnostic engine 120 can use the follow-up module 126 to prompt the user 10 to call the user's PCP or another medical or health provider, schedule an appointment with such a provider, complete an application for benefits coverage, submit a claim for benefits distribution, perform another follow-up action, or any combination thereof.

Additionally, the diagnostic engine 120 can be configured in many cases to itself perform one or more follow-up operations on behalf of the user 10 based at least in part on determining a diagnosis for the user 10. For instance, the diagnostic engine 120 can use the follow-up module 126 to call the user's PCP or another medical or health provider, schedule an appointment with such a provider, complete an application for benefits coverage, submit a claim for benefits distribution, perform another follow-up action on behalf of the user 10, or any combination thereof.

In some examples, the diagnostic engine 120 can use the follow-up module 126 to send at least one of a notification of a diagnosis determined for the user 10, an inquiry related to the diagnosis, or an appointment request based on the diagnosis to one or more of the remote computing devices 104. The diagnostic engine 120 can use the follow-up module 126 in some cases to create at least one of a reminder, a meeting, or an appointment on a digital calendar application used by the user 10 that can be accessed or modified by the follow-up module 126. The diagnostic engine 120 can also use the follow-up module 126 in other examples to implement communication between the user 10 and a third-party entity by way of at least one of the remote computing devices 104 or a communication device (e.g., telephone) associated with the third-party entity to perform one or more follow-up actions based at least in part on a diagnosis determined for the user 10. For instance, the diagnostic engine 120 can use the follow-up module 126 to implement a phone call or a video call between the user 10 and the user's PCP by way of the remote computing device 104B or a telephone of the PCP. The diagnostic engine 120 can further use the follow-up module 126 in another example to input data indicative of the user 10 and a diagnosis determined for the user 10 into data fields of a computer-generated health benefit application of a health benefit provider based at least in part on determining the diagnosis. For instance, the diagnostic engine 120 can use the follow-up module 125 to communicate with the remote computing device 104C on behalf of the user 10 to complete and submit various documentation for the user 10 in connection with applying for benefits coverage or submitting a claim for benefits distribution from the benefits provided associated with the remote computing device 104C.

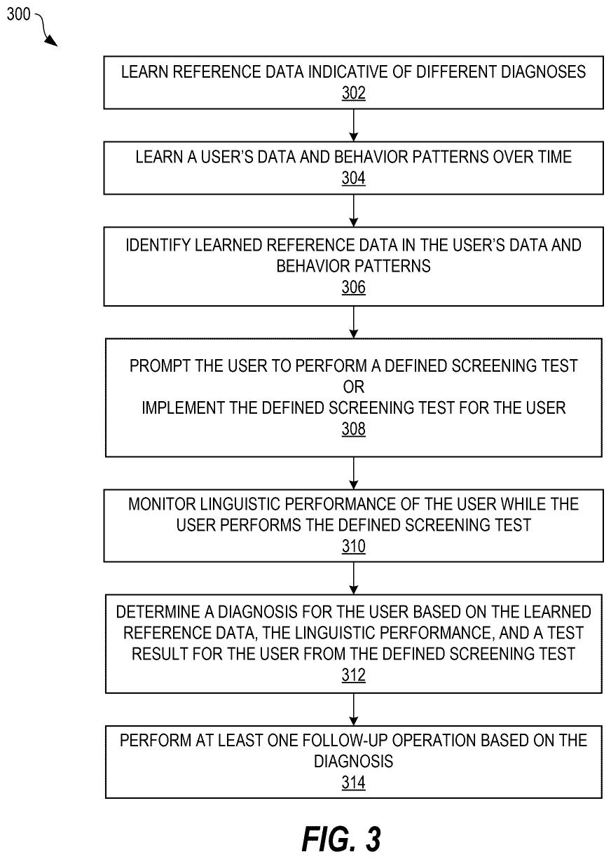

FIG. 3 illustrates a flow diagram of an example computer-implemented method 300 according to various aspects and embodiments of the present disclosure. The computer-implemented method 300 (“the method 300”) can be implemented to determine a diagnosis for a user such as the user 10 as described in examples herein. The method 300 can also be implemented to further determine at least one of severity of symptoms or onset of conditions associated with a diagnosis in some examples. The method 300 can be implemented by the computing device 102 in the context of the environment 100 using the diagnostic engine 120 as described in various examples herein.

At 302, the method 300 can include learning reference data indicative of different diagnoses. For example, as described above with reference to FIGS. 1 and 2, the computing device 102 can implement the diagnostic engine 120 to learn various reference medical and health data and information in the reference medical data sources 106A such as the reference and training data 130. For instance, the computing device 102 can implement the diagnostic engine 120 and the ML-AI models 124 to lean reference data such as reference linguistic patterns, reference digital biomarker data, reference linguistic performance data, and reference screening test results data that are at least partly indicative of different medical and health diagnosis.

At 304, the method 300 can include learning a user's data and behavior patterns over time. For example, as described above with reference to FIGS. 1 and 2, the computing device 102 can implement the diagnostic engine 120 and the ML-AI models 124 to learn the user data 132 such as medical and health data and information, linguistic patterns, digital biomarker data, linguistic performance data, and screening test results data of the user.

At 306, the method 300 can include identifying learned reference data in the user's data and behavior patterns. For example, as described above with reference to FIGS. 1 and 2, the computing device 102 can implement the diagnostic engine 120 and the ML-AI models 124 to identify reference data from the reference and training data 130 in the user data 132. For instance, the computing device 102 can implement the diagnostic engine 120 and the ML-AI models 124 to identify reference data such as a defined linguistic pattern in digital biomarker data of the user 10.

At 308, the method 300 can include prompting the user to perform a defined screening test or implementing the defined screening test for the user to complete, based at least in part on identifying the learned reference data in the user's data and behavior patterns. As described above with reference to FIGS. 1 and 2, in some examples the computing device 102 can implement the diagnostic engine 120 and the follow-up module 126 to prompt the user 10 using the I/O devices 142 to complete a defined screening test based at least in part on identifying a certain linguistic pattern in digital biomarker data of the user 10. In other examples, the computing device 102 can use the diagnostic engine 120 and the follow-up module 126 to implement a defined screening test for the user 10 to complete, based at least in part on identifying a certain linguistic pattern in digital biomarker data of the user 10. For instance, the computing device 102 (e.g., via the diagnostic engine 120 and the follow-up module 126) can implement a defined screening test for the user 10 to complete using the I/O devices 142 of the computing device 102.

The computing device 102 (e.g., via the diagnostic engine 120 and the follow-up module 126) can implement a defined screening test for the user 10 to complete in a passive manner in some cases using the I/O devices 142, with the user 10 being unaware of the test and the fact that the user 10 is taking the test. The computing device 102 can implement a defined screening test for the user 10 to complete in an active manner in other cases using the I/O devices 142, with the user 10 being aware of the test and the fact that the user 10 is taking the test.

In some examples, the computing device 102 (e.g., via the diagnostic engine 120 and the follow-up module 126) can implement a defined screening test such as a verbal fluency activity, process, or test that is to be completed by the user 10 in a passive or active manner using the I/O devices 142 of the computing device 102. In one example, the computing device 102 can implement a defined screening test such as a verbal fluency test that can be completed by the user 10 by way of the user 10 reciting certain content aloud such as one or more words, numbers, phrases, sentences, paragraphs, monologs, excerpts, or some other content the user 10 can verbally recite. In another example, the computing device 102 can implement a defined screening test such as a verbal fluency test that can be completed by the user 10 by way of the user 10 having a conversation with a third-party entity (e.g., an individual, a trained ML or AI model). For instance, the computing device 102 can implement a verbal fluency test while the user 10 is using the computing device 102 to have a conversation with a third-party entity (e.g., during a phone call between the user 10 and the third-party entity). In another example, the computing device 102 can implement a verbal fluency test while the user 10 is having a conversation with a third-party entity in the presence of (e.g., physically near or adjacent) the computing device 102.

In any case where verbal fluency test data for the user 10 is captured during a conversation between the user 10 and a third-party entity as described herein, the computing device 102 can prevent the capture and recording of any data pertaining to the third-party entity. For instance, the computing device 102 (e.g., via the diagnostic engine 120 and the follow-up module 126) can be configured to only capture and record verbal fluency test data of the user 10 and to delete any data of a third-party entity that has been inadvertently captured such as during completion of a defined screening test by the user 10.

At 310, the method 300 can include monitoring linguistic performance of the user while the user performs the defined screening test. For example, as described above with reference to FIGS. 1 and 2, the computing device 102 can implement the diagnostic engine 120, the data collection module 122, the sensors 140, and the I/O devices 142 to capture and analyze various data from the user 10 while the user 10 completes the defined screening test such as when the user 10 is speaking or moving during the test.

In any case where linguistic performance data or other data for the user 10 is captured while the user 10 is performing a defined screening test, the computing device 102 can prevent the capture and recording of any data pertaining to a third-party entity. For instance, the computing device 102 (e.g., via the diagnostic engine 120 and the follow-up module 126) can be configured to only capture and record linguistic performance data of the user 10 and to delete any data of a third-party entity that has been inadvertently captured with such linguistic performance data of the user 10.

At 312, the method 300 can include determining a diagnosis for the user based at least in part on the learned reference data, the linguistic performance, and a test result for the user from the defined screening test. For example, the computing device 102 can implement the diagnostic engine 120 and the ML-AI models 124 to determine a diagnosis for the user 10 based at least in part on identifying a certain linguistic pattern in the user data 132, linguistic performance of the user 10 captured while the user 10 performs a defined screening test, and a test result for the user 10 obtained upon completion of the defined screening test.

At 314, the method 300 can include performing at least one follow-up operation based at least in part on a diagnosis determined for the user. For example, as described above with reference to FIGS. 1 and 2, the computing device 102 can implement the diagnostic engine 120, the follow-up module 126, and the I/O devices 142 to prompt the user 10 to perform at least one follow-up action based in part on a diagnosis such as calling or making an appointment with a medical provider, completing and submitting benefits related documentation, or another action. The computing device 102 can implement the diagnostic engine 120 in other cases to itself perform such a follow-up action on behalf of the user 10 based at least in part on a diagnosis determined for the user 10.

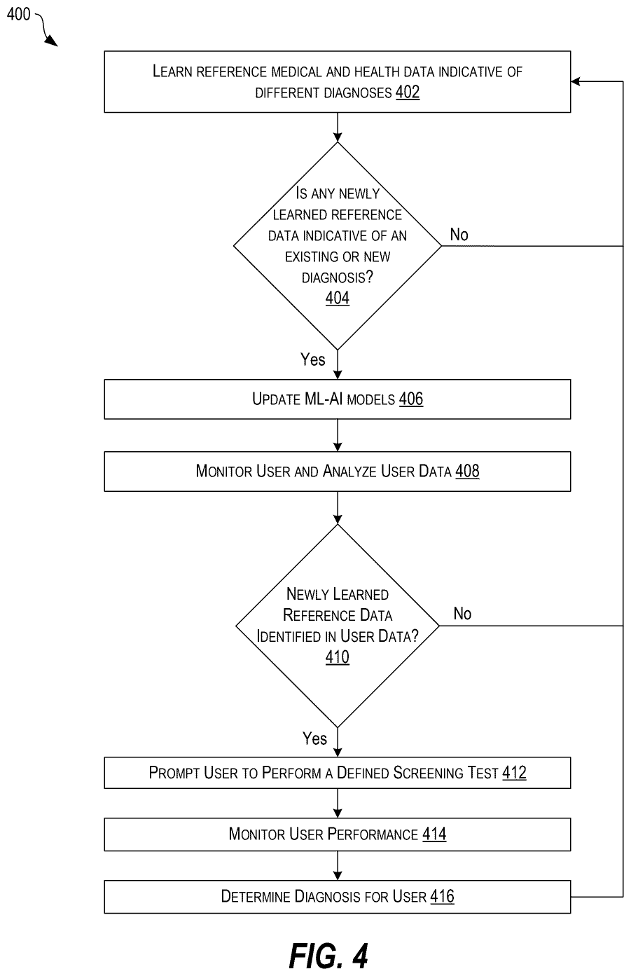

FIG. 4 illustrates a flow diagram of another example computer-implemented method 400 according to various aspects and embodiments of the present disclosure. The computer-implemented method 400 (“the method 400”) can be implemented to determine a diagnosis for a user such as the user 10 as described in examples herein. The method 400 can also be implemented to further determine at least one of severity of symptoms or onset of conditions associated with a diagnosis in some examples. The method 400 can be implemented by the computing device 102 in the context of the environment 100 using the diagnostic engine 120 as described in various examples herein.

At 402, the method 400 can include continuously learning new or updated reference medical and health data indicative of different existing or new diagnoses. For example, the computing device 102 can implement the diagnostic engine 120 and the ML-AI models 124 to continuously learn various new or updated reference medical and health data and information in the reference medical data sources 106A such as new or updated reference medical and health data. For instance, the computing device 102 can implement the diagnostic engine 120 and the ML-AI models 124 to continuously lean new or updated reference data such as new or updated reference linguistic patterns, new or updated reference digital biomarker data, new or updated reference linguistic performance data, and new or updated reference screening test results data that are at least partly indicative of different existing or new medical and health diagnosis.

At 404, the method 400 can include determining whether any newly learned reference data learned at 402 are indicative of an existing or new diagnosis. If it is determined at 404 that the newly leaned reference data are not indicative of an existing or new diagnosis, then the method 400 returns to and repeats 402. If it is determined at 404 that the newly leaned reference data are indicative of an existing or new diagnosis, then the method 400 at 406 can include updating the ML-AI models 124 to account for the newly learned reference data indicative of an existing or new diagnosis.

At 408, the method 400 can include monitoring a user and analyzing the user's data. For example, the computing device 102 can implement the diagnosis engine 120, the data collection module 122, updated versions of the ML-AI models 124, the sensors 140, and the I/O devices 142 to monitor the user 10, collect the user data 132, and analyze such data relative to the newly learned reference data learned at 402.

At 410, the method 400 can include determining whether any newly learned reference data learned at 402 are identified in the user's data such as the user data 132. If it is determined at 410 that there are not any newly leaned reference data identified in the user's data, then the method 400 returns to and repeats 402. If it is determined at 410 that there are newly leaned reference data in the user's data, then the method 400 can include prompting the user to perform a defined screening test at 412, monitoring the user's performance while the user completes the defined screening test at 414, and determining a diagnosis for the user at 416. The computing device 102 can implement the diagnostic engine 120 as described with reference to FIGS. 1, 2, and 3 to perform the operations at 410, 412, 414, and 416 of the method 400, at which point the method 400 returns to and repeats 402.

Referring now to FIG. 2, an executable program can be stored in any portion or component of the memory 114. The memory 114 can be embodied as, for example, a random access memory (RAM), read-only memory (ROM), magnetic or other hard disk drive, solid-state, semiconductor, universal serial bus (USB) flash drive, memory card, optical disc (e.g., compact disc (CD) or digital versatile disc (DVD)), floppy disk, magnetic tape, or other types of memory devices.

The memory 114 can include both volatile and nonvolatile memory and data storage components. Volatile components are those that do not retain data values upon loss of power. Nonvolatile components are those that retain data upon a loss of power. Thus, the memory 114 can include, for example, a RAM, ROM, magnetic or other hard disk drive, solid-state, semiconductor, or similar drive, USB flash drive, memory card accessed via a memory card reader, floppy disk accessed via an associated floppy disk drive, optical disc accessed via an optical disc drive, magnetic tape accessed via an appropriate tape drive, and/or other memory component, or any combination thereof. In addition, the RAM can include, for example, a static random-access memory (SRAM), dynamic random-access memory (DRAM), or magnetic random-access memory (MRAM), and/or other similar memory device. The ROM can include, for example, a programmable read-only memory (PROM), erasable programmable read-only memory (EPROM), electrically erasable programmable read-only memory (EEPROM), or other similar memory devices.

As discussed above, the diagnostic engine 120, the data collection module 122, the ML-AI models 124, the follow-up module 126, and the communications stack 128 can each be embodied, at least in part, by software or executable-code components for execution by general purpose hardware. Alternatively, the same can be embodied in dedicated hardware or a combination of software, general, specific, and/or dedicated purpose hardware. If embodied in such hardware, each can be implemented as a circuit or state machine, for example, that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components.

Referring now to FIGS. 3 and 4, the flowchart or process diagram shown in each of FIGS. 3 and 4 is representative of certain processes, functionality, and operations of the embodiments discussed herein. Each block can represent one or a combination of steps or executions in a process. Alternatively, or additionally, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as the processor 112. The machine code can be converted from the source code. Further, each block can represent, or be connected with, a circuit or a number of interconnected circuits to implement a certain logical function or process step.

Although the flowchart or process diagram shown in each of FIGS. 3 and 4 illustrates a specific order, it is understood that the order can differ from that which is depicted. For example, an order of execution of two or more blocks can be scrambled relative to the order shown. Also, two or more blocks shown in succession can be executed concurrently or with partial concurrence. Further, in some embodiments, one or more of the blocks can be skipped or omitted. In addition, any number of counters, state variables, warning semaphores, or messages might be added to the logical flow described herein, for purposes of enhanced utility, accounting, performance measurement, or providing troubleshooting aids. Such variations, as understood for implementing the process consistent with the concepts described herein, are within the scope of the embodiments.

Also, any logic or application described herein, including the diagnostic engine 120, the data collection module 122, the ML-AI models 124, the follow-up module 126, and the communications stack 128 can be embodied, at least in part, by software or executable-code components, can be embodied or stored in any tangible or non-transitory computer-readable medium or device for execution by an instruction execution system such as a general-purpose processor. In this sense, the logic can be embodied as, for example, software or executable-code components that can be fetched from the computer-readable medium and executed by the instruction execution system. Thus, the instruction execution system can be directed by execution of the instructions to perform certain processes such as those illustrated in FIGS. 3 and 4. In the context of the present disclosure, a non-transitory computer-readable medium can be any tangible medium that can contain, store, or maintain any logic, application, software, or executable-code component described herein for use by or in connection with an instruction execution system.

The computer-readable medium can include any physical media such as, for example, magnetic, optical, or semiconductor media. More specific examples of suitable computer-readable media include, but are not limited to, magnetic tapes, magnetic floppy diskettes, magnetic hard drives, memory cards, solid-state drives, USB flash drives, or optical discs. Also, the computer-readable medium can include a RAM including, for example, an SRAM, DRAM, or MRAM. In addition, the computer-readable medium can include a ROM, a PROM, an EPROM, an EEPROM, or other similar memory device.

Disjunctive language, such as the phrase “at least one of X, Y, or Z,” unless specifically stated otherwise, is to be understood with the context as used in general to present that an item, term, or the like, can be either X, Y, or Z, or any combination thereof (e.g., X, Y, and/or Z). Thus, such disjunctive language is not generally intended to, and should not, imply that certain embodiments require at least one of X, at least one of Y, or at least one of Z to be each present. As referenced herein in the context of quantity, the terms “a” or “an” are intended to mean “at least one” and are not intended to imply “one and only one.”

As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling), mechanical coupling, operative coupling, optical coupling, and/or physical coupling.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

1. A computer-implemented method of diagnosing health conditions for users, the method comprising:
identifying, by a computing device, a defined linguistic pattern in digital biomarker data of a user;
prompting, by the computing device, the user to perform a defined screening test based at least in part on identifying the defined linguistic pattern;
monitoring, by the computing device, linguistic performance of the user while the user performs the defined screening test; and
determining, by the computing device, a diagnosis for the user based at least in part on the defined linguistic pattern, the linguistic performance, and a test result for the user from the defined screening test.

2. The method of claim 1, further comprising:
capturing, by the computing device, the digital biomarker data of the user while the user performs an activity,
wherein the digital biomarker data comprise at least one of audio data of the user speaking or moving, video data of the user speaking or moving, image data of the user speaking or moving, textual data typed by the user, or physiological data of the user captured while the user speaks or moves.

3. The method of claim 1, further comprising:
learning, by the computing device, a plurality of linguistic patterns of the user over time based at least in part on historical digital biomarker data collected from the user at different times and during different activities.

4. The method of claim 1, further comprising:
learning, by the computing device, reference medical data that are at least partly indicative of the diagnosis,
wherein the reference medical data comprise at least one of reference linguistic patterns, reference digital biomarker data, reference linguistic performance data, or reference screening test result data that are at least partly indicative of the diagnosis.

5. The method of claim 1, wherein monitoring the linguistic performance of the user comprises:
analyzing, by the computing device, at least one of audio data, video data, or physiological data of the user that are captured while the user performs the defined screening test.

6. The method of claim 1, wherein determining the diagnosis comprises:
comparing, by the computing device, linguistic patterns of the user learned over time to reference linguistic patterns that are at least partly indicative of the diagnosis;
comparing, by the computing device, the digital biomarker data to reference digital biomarker data that are at least partly indicative of the diagnosis;
comparing, by the computing device, the linguistic performance to reference linguistic performance data that are at least partly indicative of the diagnosis; and
comparing, by the computing device, the test result to reference screening test result data that are at least partly indicative of the diagnosis.

7. The method of claim 1, further comprising at least one of:
determining, by the computing device, severity of one or more symptoms associated with the diagnosis; or
predicting, by the computing device, an onset of one or more conditions associated with the diagnosis.

8. The method of claim 1, further comprising at least one of:
prompting, by the computing device, the user to perform one or more follow-up actions based at least in part on the diagnosis.

9. The method of claim 1, further comprising at least one of:
performing, by the computing device, at least one follow-up operation based at least in part on the diagnosis.

10. The method of claim 1, further comprising:
sending, by the computing device, a notification of the diagnosis to a second computing device associated with at least one of a caretaker, a health provider, or a health benefit provider based at least in part on determining the diagnosis.

11. The method of claim 1, further comprising:
implementing, by the computing device, communication between the user and a third-party entity by way of at least one of a third-party computing device or communication device associated with the third-party entity to perform one or more follow-up actions based at least in part on the diagnosis,
wherein the third-party entity comprises at least one of a caretaker, a health provider, or a health benefit provider.

12. The method of claim 1, further comprising:
inputting, by the computing device, data indicative of the user and the diagnosis into data fields of a computer-generated health benefit application of a health benefit provider based at least in part on determining the diagnosis.

13. A computing device, comprising:
a memory device to store computer-readable instructions thereon; and
at least one processing device configured through execution of the computer-readable instructions to:
identify a defined linguistic pattern in digital biomarker data of a user;
prompt the user to perform a defined screening test based at least in part on identifying the defined linguistic pattern;
monitor linguistic performance of the user while the user performs the defined screening test; and
determine a diagnosis for the user based at least in part on the defined linguistic pattern, the linguistic performance, and a test result for the user from the defined screening test.

14. The computing device of claim 13, wherein the at least one processing device is further configured to:
capture the digital biomarker data of the user while the user performs an activity,
wherein the digital biomarker data comprise at least one of audio data of the user speaking or moving, video data of the user speaking or moving, image data of the user speaking or moving, textual data typed by the user, or physiological data of the user captured while the user speaks or moves.

15. The computing device of claim 13, wherein the at least one processing device is further configured to:
learn a plurality of linguistic patterns of the user over time based at least in part on historical digital biomarker data collected from the user at different times and during different activities.

16. The computing device of claim 13, wherein the at least one processing device is further configured to:
learn reference medical data that are at least partly indicative of the diagnosis,
wherein the reference medical data comprise at least one of reference linguistic patterns, reference digital biomarker data, reference linguistic performance data, or reference screening test result data that are at least partly indicative of the diagnosis.

17. The computing device of claim 13, wherein to identify the defined linguistic pattern, the at least one processing device is further configured to:
compare linguistic patterns of the user learned over time to reference linguistic patterns that are at least partly indicative of the diagnosis;
compare the digital biomarker data to reference digital biomarker data that are at least partly indicative of the diagnosis;
compare the linguistic performance to reference linguistic performance data that are at least partly indicative of the diagnosis; and
compare the test result to reference screening test result data that are at least partly indicative of the diagnosis.

18. The computing device of claim 13, wherein the at least one processing device is further configured to:
determine severity of one or more symptoms associated with the diagnosis; or
predict an onset of one or more conditions associated with the diagnosis.

19. The computing device of claim 13, wherein the at least one processing device is further configured to:
prompt the user to perform one or more follow-up actions based at least in part on the diagnosis; or
perform at least one follow-up operation based at least in part on the diagnosis.

20. A computer-implemented method of diagnosing health conditions for users, the method comprising:
identifying, by a computing device, a defined linguistic pattern in digital biomarker data of a user;
implementing, by the computing device, a defined screening test to be performed by the user based at least in part on identifying the defined linguistic pattern;
monitoring, by the computing device, linguistic performance of the user during the defined screening test; and
determining, by the computing device, a diagnosis for the user based at least in part on the defined linguistic pattern, the linguistic performance, and a test result for the user from the defined screening test.
